(19) United States
(12) Patent Application Publication
(10) Pub. No.: US 2025/0152119 A1
(43) Pub. Date: May 15, 2025

(54) WALL MOTION ABNORMALITY DETECTION VIA AUTOMATED EVALUATION OF VOLUME RENDERING MOVIES

(71) Applicant: The Regents of the University of California, Oakland, CA (US)

(72) Inventors: Francisco Contijoch, San Diego, CA (US); Zhennong Chen, La Jolla, CA (US); Elliot McVeigh, San Diego, CA (US)

(73) Assignee: The Regents of the University of California, Oakland, CA (US)

(21) Appl. No.: 18/834,619

(22) PCT Filed: Feb. 2, 2023
(86) PCT No.: PCT/US2023/061885

§ 371 Date: Jul. 30, 2024

Related U.S. Application Data
(60) Provisional application No. 63/267,479, filed on Feb. 2, 2022.

(51) Int. Cl. A61B 6/00; A61B 6/03; A61B 6/46; A61B 6/50; G06T 7/00; G06T 7/246; G06T 15/08; G16H 10/60; G16H 30/40; G16H 50/20

(52) U.S. Cl. CPC A61B 6/5217; A61B 6/03; A61B 6/463; A61B 6/486; A61B 6/503; G06T 7/0012; G06T 7/246; G06T 15/08; G16H 10/60; G16H 30/40; G16H 50/20; G06T 2207/10016; G06T 2207/10024; G06T 2207/10081; G06T 2207/20081; G06T 2207/20084; G06T 2207/30048; G06T 2207/30101; G06T 2210/41

(57) ABSTRACT

Systems and methods that pertain to a cardiac function abnormality detection via automated evaluation of volume rendering movies are disclosed. In some implementations, a system includes a view generator to create a plurality of volume rendered views of an organ of a patient, a motion detector coupled to the view generator to detect a regional motion of a section of the organ based on the plurality of volume rendered views of the organ, and a display coupled to the motion detector to show the plurality of volume rendered views or a detection of an abnormality of the section.

### CROSS-REFERENCE TO RELATED APPLICATION

This patent document claims priority to and benefits of U.S. Provisional Appl. No. 63/267,479, entitled “WALL MOTION ABNORMALITY DETECTION VIA AUTOMATED EVALUATION OF VOLUME RENDERING MOVIES” and filed on Feb. 2, 2022. The entire contents of the before-mentioned patent application are incorporated by reference as part of the disclosure of this document.

### STATEMENT REGARDING FEDERALLY SPONSORED RESEARCH OR DEVELOPMENT

This invention was made with government support under HL143113, and HL144678 awarded by the National Institutes of Health. The government has certain rights in the invention.

### TECHNICAL FIELD

The disclosed technology relates to diagnosis for cardiac wall motion abnormalities in human heart.

### BACKGROUND

Cardiac wall motion abnormalities such as left ventricular (LV) wall motion abnormalities (WMA) have both diagnostic and prognostic significance in patients with heart disease. 4D imaging methods such as multi-detector cine 4D computed tomography (CT), 4D cardiac MRI, and 3D cardiac echocardiography are increasingly used to evaluate cardiac function. The clinical WMA assessment from 4D imaging is usually limited to viewing the re-formatted 2D short-axis and long-axis imaging planes. However, this only contains partial information about the complex 3D wall motion.

### SUMMARY

The disclosed technology can be implemented in some embodiments to provide methods, materials and devices that can automatically detect cardiac wall motion abnormalities in human heart.

In some implementations of the disclosed technology, a system includes a view generator to create a plurality of volume rendered views of an organ of a patient, a motion detector coupled to the view generator to detect a regional motion of a section of the organ based on the plurality of volume rendered views of the organ, and a display coupled to the motion detector to show the plurality of volume rendered views or a detection of an abnormality of the section.

In some implementations of the disclosed technology, a system includes a view generator to create a plurality of volume rendered views of an organ of a patient, a motion detector coupled to the view generator and including: a first network to extract spatial features from each input frame of the plurality of volume rendered views of the organ; a second network to extract temporal information from a sequence of volume rendered frames corresponding to the plurality of volume rendered views of the organ; and an algorithm to classify a severity of a motion abnormality of the organ, and a display coupled to the motion detector to show the severity of the motion abnormality of the organ by assigning different colors to different levels of the severity of the motion abnormality of the organ.

In some implementations of the disclosed technology, a method for detecting heart disease in a patient includes obtaining a plurality of volume rendering videos from cardiac imaging data of the patient, classifying cardiac wall motion abnormalities present in the plurality of volume rendering videos, and determining whether the cardiac wall motion abnormalities in the volume rendering videos are associated with the heart disease of the patient.

The above and other aspects and implementations of the disclosed technology are described in more detail in the drawings, the description and the claims.

### BRIEF DESCRIPTION OF THE DRAWINGS

FIG. 1 shows an example of automatic generation of volume rendering (VR) video based on some embodiments of the disclosed technology.

FIG. 2 shows an example of deep learning network implemented based on some embodiments of the disclosed technology.

FIG. 3 shows automatic generation and quantitative labeling of volume rendering video based on some embodiments of the disclosed technology.

FIG. 4 shows the relationship between DL classification accuracy and left ventricular ejection fraction (LVEF) in the cross-validation.

FIG. 5 shows an example system 500 implemented based on some embodiments of the disclosed technology.

FIG. 6 is a flow diagram that illustrates an example method 600 for detecting a heart disease of a patient based on some embodiments of the disclosed technology.

### DETAILED DESCRIPTION

Section headings are used in the present document only for ease of understanding and do not limit scope of the embodiments to the section in which they are described.

Disclosed are systems and methods that pertain to a wall motion abnormality detection via direct visualization and/or automated evaluation of volume rendering movies. The invention relates to methods and devices that can automatically detect cardiac wall motion abnormalities in human heart.

Multi-detector cine 4D computed tomography (CT) is one embodiment of 4D cardiac data collection; 4D CT is increasingly used to evaluate cardiac function. The clinical WMA assessment from CT and other modalities are usually limited to viewing the re-formatted 2D short-axis and long-axis imaging planes. However, this only contains partial information about the complex 3D wall motion. While 3D feature tracking approaches have been developed to capture this complex deformation, algorithms typically require manipulating the 4D dataset. The large size of the 4DCT data also limits the use of deep-learning (DL) algorithms to automatically detect the 3D WMA from 4DCT studies as current graphics processing units (GPU) do not have the capacity to take multiple frames of 4DCT (˜2 Gigabyte) as the input.

The disclosed technology can be implemented in some embodiments to provide a deep-learning (DL)-based framework that automatically detects cardiac motion abnormalities such as wall motion abnormalities (WMAs) from volume rendering (VR) videos of clinical cardiac 4D data such as computed tomography (CT), MRI, or echocardiography studies. VR video provides a highly representative and memory efficient (e.g., ˜300 Kilobyte) way to visualize the entire complex cardiac wall motion such as 3D left ventricular (LV) wall motion efficiently and coherently. In some implementations, an automated process generates VR videos from clinical 4D data and then a neural network is trained to detect WMA from VR video as inputs.

Subtle motion abnormalities in heart contraction dynamics can be directly observed on movies of 3D volumes obtained from imaging modalities such as computed tomography (CT). The high resolution views of endocardial 3D topological features in 4D CT are not available from any other clinical imaging strategy. Experimentally, direct intra cardiac camera views can be obtained after the blood is replaced with transparent fluid; however, this is not done clinically. High spatial resolution views of large segments of the deforming endocardium are available from volume rendered CT, and clearly show detailed definition of abnormal regions, but the power of these images as quantitative diagnostic tools has not been developed to date. This is a completely unappreciated opportunity-principally because the amount of data used to create the movies is too cumbersome for daily use on scanners and departmental picture archiving systems, so the method of direct analysis of dynamic 4D data has gone undeveloped.

The disclosed technology can be implemented in some embodiments to provide a display system in which volume rendered views of chambers of the heart are created to directly detect regional myocardial wall motion details visually by an observer, or be detected automatically via any image processing algorithm (such as a deep learning network) applied directly to the movies such that: (1) the observer detects regional functional abnormalities; (2) the observer detects the size, shape, border zone of an infarct or other regional abnormalities; and/or (3) the observer detects a change in cardiac function during stress. In some implementations, the display system for echocardiography is commonly used in current clinical practice.

In some embodiments of the disclosed technology, a display system includes a view generator to create a plurality of volume rendered views of an organ of a patient, a motion detector to detect a regional motion of a section of the organ based on the plurality of volume rendered views of the organ, and a display coupled to the motion detector to show the plurality of volume rendered views or a detection of an abnormality of the section using an image processing algorithm.

In some implementations, the section of the organ includes a heart chamber of the patient. In some implementations, the section of the organ includes a myocardial wall of the patient. In some implementations, the image processing algorithm includes a deep learning network. In some implementations, the abnormality includes regional ischemia or regional infarction. In some implementations, the abnormality includes a change in a left ventricular (LV) function. In some implementations, the volume rendered views include at least one of size, shape, or border zone of a myocardial infarction.

CT is becoming more common in cardiology clinical practice due to recent data showing it yields the best data for predicting future cardiovascular events and response to intervention. As the number of patients who undergo cardiac CT increases, this method for evaluating myocardial wall motion will become widely available.

Four-dimensional computed tomography (4DCT) movies are not commonly reconstructed on clinical scanners due to memory limitations on the scanner and on the departmental picture archiving system. CT images are large 3D volumes (usually 512×512×256 voxels). They can be acquired as 4D dynamic data movies spanning the cardiac cycle which leads to a 4D dataset which is larger than a single 3D image (by a factor of 10 to 20), yielding approximately about 2 Gb of data per case. As a result, interpretation usually requires expensive servers and advanced visualization software which is not common in most clinical departments.

To evaluate if a part of the heart is not contracting normally, physicians either look at the motion or thickening of different parts of the heart. A quantitative estimate of function is usually obtained in the clinic by tracing the boundaries of the heartwall and measuring changes in myocardial wall thickness during the cardiac cycle. This method is time consuming and susceptible to user to user variability.

The volume rendered approach based on some embodiments of the disclosed technology can avoid these difficulties/challenges. By representing the heart motion using volume rendering we can observe cardiac function abnormalities and wall motion abnormalities directly, either by direct viewing or using an image processing/machine-learning framework. By performing volume rendering from different perspectives, different portions (e.g., different LV walls) of the heart can be analyzed and the whole patient can be assessed.

Volume renderings are very memory efficient (˜500-1000 fold compression over the original 4D data) and the display system based on some embodiments of the disclosed technology can accurately classify patients as being normal or abnormal using the approach discussed in this patent document.

In some implementations, the display system can include a machine-learning algorithm to look at a series of the images of the movies generated from the 4D data and determine whether it is a normal or abnormal pattern of contraction, and estimate the severity of the abnormality.

The disclosed technology can be implemented in some embodiments to visualize 3D features over a large section of the heart, or heart wall, unlike other clinical imaging modalities.

Existing CT methods have relied on wall thickness measurements in 2D slices which provide point-wise measurement of function. In addition to defining the endocardial boundary, this requires tracing the epicardial boundary. Thickness is also affected by the direction of the measurement so the 3D orientation of the measurement matters.

In some embodiments of the disclosed technology, once represented as volume renderings, the size of the dataset analyzed is significantly reduced. This enables efficient training for machine learning, such as a neural network for detecting and quantifying abnormalities.

The approach based on some embodiments of the disclosed technology includes training a neural network on sequences of volume rendered images.

The disclosed technology can be implemented in some embodiments to provide a program by which a set of images acquired in a patient can be analyzed on the scanner in a few seconds after image reconstruction to assess whether one of their heart walls is moving abnormally. Some embodiments of the disclosed technology can be used to confirm coronary artery disease detected by visual assessment by the physician. Some embodiments of the disclosed technology can also be used to identify coronary vessels as being likely obstructed (and guide the visual interpretation). Some embodiments of the technology can outline the boundaries of an abnormality such as regional ischemia, or infarction. Some embodiments of the technology can define the “border zone” of myocardial infarction. Some embodiments of the disclosed technology can replace almost all uses of echocardiography than involve perceiving wall motion.

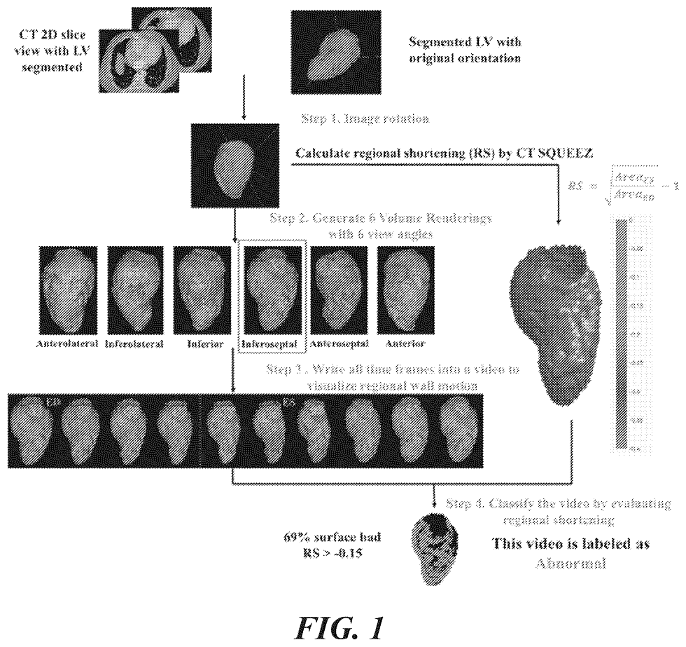

FIG. 1 shows an example of automatic generation of volume rendering (VR) video based on some embodiments of the disclosed technology.

Referring to FIG. 1, each CT scan generates 6 VR with 6 view angles. In step 2, the myocardial wall in the foreground is noted under each view. The bottom row of FIG. 1 shows frames from a VR video example with the inferoseptal region of the LV wall in the foreground, which is labeled as abnormal according to a regional myocardial shortening calculation.

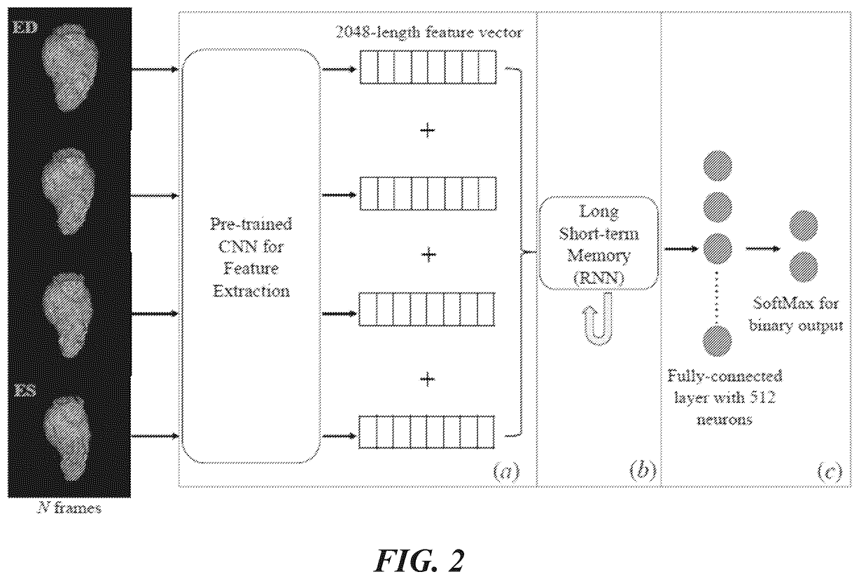

FIG. 2 shows an example of a deep learning network implemented based on some embodiments of the disclosed technology.

Referring to FIG. 2, N (=4 in this figure) frames are input individually into component (a), a pre-trained convolutional neural network (CNN) for image feature extraction. Feature vectors are concatenated into a sequence and input into component (b), a recurrent neural network (RNN). Component (c), a fully-connected neural network logistically regresses the binary classification of the wall motion abnormalities (WMA) presence/absence in the video of volume rendered views. Cardiac wall motion abnormalities such as left ventricular (LV) wall motion abnormalities (WMA) have both diagnostic and prognostic significance in patients with heart disease. Multi-detector cine 4D computed tomography (CT) is increasingly used to evaluate cardiac function. The clinical WMA assessment from CT is usually limited to viewing the re-formatted 2D short- and long-axis imaging planes. However, this only contains partial information about the complex 3D wall motion. While 3D feature tracking approaches have been developed to capture this complex deformation but algorithms typically require manipulating the 4D dataset. The large size also limits the use of deep-learning (DL) algorithms to automatically detect the 3D WMA from 4DCT studies as current graphics processing units (GPU) do not have the capacity to take multiple frames of 4DCT (˜2 Gigabyte) as the input.

The disclosed technology can be implemented in some embodiments to provide a novel DL-based framework that automatically detects WMAs from Volume Rendering (VR) videos of clinical cardiac CT studies. VR video provides a highly representative and memory efficient (˜300 Kilobyte) way to visualize the entire complex 3D LV wall motion efficiently and coherently. We defined an automated process to generate VR videos from clinical 4DCT data and then trained a neural network to detect WMA from VR video as inputs.

We retrospectively evaluated 253 cardiac CT studies for DL training and testing. VR videos were automatically generated for each study. The DL framework consists of a pre-trained convolutional neural network (CNN) and a recurrent neural network (RNN) trained to predict the presence of WMA from each VR video. CT Data Collection and Image Preprocessing

In some implementations, 253 ECG-gated contrast enhanced cardiac CT patient studies were retrospectively collected for DL training/testing with IRB approval with waiver of informed consent. Each study had images reconstructed across the entire cardiac cycle and had a field-of-view which captured the entire LV. Images were collected on a single, wide detector CT scanner with 256 rows allowing for a single heartbeat axial 16 cm acquisition throughout the cardiac cycle. The CT studies were performed for range of clinical cardiac indications including suspected coronary artery disease (n=106), pre-operative assessment of pulmonary vein ablation (n=105), evaluation for transcatheter aortic valve replacement (n=27), and evaluation for ventricular assist device placement (n=15).

Pixel-wise segmentation of LV blood-pool was first predicted by a pre-trained convolutional neural network architecture (e.g., 2D U-Net) and then refined by a cardiovascular imaging expert. Segmented images were then rotated so that the long axis of the LV corresponded with the z-axis.

### Automated Volume Rendering Video Generation

Volume rendering (VR) of the LV blood pool in pre-processed images was created automatically using a built-in function (e.g., “volshow” in MATLAB). VR assigned different colors and opacities to each pixel according to its intensity. The study-specific window level used for rendering was determined based on the mean attenuation of the LV blood-pool and the window width was 150 HU for all studies. VR of all frames spanning one cardiac cycle is then written into a video.

One VR video shows the LV blood volume from one specific view angle. To evaluate all LV walls, 6 VR videos were generated per study, at sequential 60-degree rotations around the LV long axis (see FIG. 1). In total, 1518 VR videos (253 patients×6 views) were generated.

### Classification of WMA Presence in Volume Rendering Video

Ground truth binary classification of the presence or absence of wall motion abnormalities (WMA) can be determined for each VR video by quantitatively evaluating the extent of impaired 3D regional shortenings (RS_{CT}) of the endocardium associated with the VR video view. A 4D endocardial surface feature tracking algorithm that has been previously validated with tagged MRI for measuring regional myocardial function can be used. RS_{CT} of the endocardial surface between end-diastole and end-systole (ED and ES, defined in each video as the frame with the largest and smallest LV endocardial volume correspondingly) can be calculated based on:

$$RS ( p , ES ) = \sqrt{\frac{{A}_{( p , ES )}}{{A}_{( p , ED )}}} - 1$$

where A is the area of a triangular mesh associated with point p on the endocardium. RS_{CT} values can be projected based on each VR video view. A VR video was classified as abnormal (WMA present) if more than 30% of the endocardial surface includes impaired RS_{CT}(>−0.20). The 30% and −0.20 thresholds were chosen empirically. The classification results can be visually confirmed by an expert reader. A CT scan (which consists of 6 VR videos) can be classified as abnormal if more than one video is classified as abnormal.

### Deep Learning Data Split

The dataset was split chronologically into two cohorts. The training cohort contained all CT studies from January 2018 to December 2019 (174 studies, 1044 videos). The training cohort was randomly and equally split into five groups for 5-fold cross-validation. We report model performance across all folds. The testing cohort contained all independent studies from January 2020 to June 2020 (79 studies, 474 videos) and was used to evaluate the model.

### Deep Learning Framework Design

As shown in FIG. 2, the deep learning (DL) framework based on some embodiments of the disclosed technology includes three components: (a) a pre-trained convolutional neural network (CNN) used to extract spatial features from each input frame of a VR video; (b) a recurrent neural network (RNN) designed to synthesize the temporal relationship between frames; (c) a fully connected neural network designed to output the classification. In some example implementations that focus on systolic function, N systolic frames may be input to the DL framework. In one example, component (a) creates a 2048-length feature vector for each input frame individually. Feature vectors from N frames are then concatenated into a feature sequence with size=(N,2048). In one example, component (b) is a RNN that includes a long short-term memory architecture with 2048 nodes and sigmoidal activation function. This RNN takes the feature sequence from component (a) and incorporated the temporal relationship. The final component (c) logistically regresses the binary prediction of the presence of WMA in the VR video.

In the DL framework implemented based on some embodiments of the disclosed technology, component (a) is pre-trained and directly used for feature extraction whereas components (b) and (c) are trained end-to-end as one network. The loss function is categorical cross-entropy.

### Model Tuning

The model tuning is twofold: the choices of different model architecture for component (a) and the choices of different N, the number of systolic frames of the video input into the framework. For architecture, CNNs with the highest top-1 accuracy in the ImageNet validation dataset available in Keras Applications (e.g., Xception, ResNet152V2, Inception V3, InceptionResNetV2) can be used. All pre-trained models can use layers up to the average pooling layer to output a feature vector. Only InceptionResNetV2 outputs a 1536-length vector (thus the nodes of RNN can be adapted) while the rest of the networks (Xception, ResNet152V2, Inception V3) output 2048-length vectors. In some implementations, for the choice of number of timeframes N, since the earliest end-systolic frame in this dataset is the fourth frame, the Nis chosen to be 2 (ED and ES frames), 3 (ED, ES and mid-systole frames) and 4 (ED, ES, and two systolic frames with equal gaps). In some implementations, all 12 (4 architectures×3 choices for number of frames) are trained on 80% of the training cohort and validated on the rest 20%. In some implementations, the combination with the highest per-video validation accuracy is picked as the final design.

### Experiment Setup

We performed all DL experiments by using Keras with TensorFlow a workstation. The times needed to train the framework and to predict on new cases were recorded. The file size of each CT study as well as each generated VR video was also recorded.

### Statistical Evaluation

The DL performance was evaluated against the ground truth labels in terms of per-video and per-study accuracy, sensitivity, and specificity. Two-tailed categorical z-test was used to evaluate the difference of data composition (e.g., the percentage of abnormal videos) and the difference of model performance (e.g., accuracy) between the training cohort and testing cohort. Statistical significance was set at P≤0.05.

### Results

In the training cohort, 107 (61.5%) were male (age: 61±15) and 67 (38.5%) were female (age: 64±14). The LV blood-pool had a median intensity of 516 HU (IQR: 433 to 604). 34.9% (364/1044) of the videos were labeled as abnormal, and 41.4% (72/174) of CT studies were defined as abnormal study (had>1 abnormal videos).

In the testing cohort, 46 (58.2%) were male (age: 62±13) and 33 (41.8%) were female (age: 61±14). The LV blood-pool had a median intensity of 507 HU (IQR: 444 to 483). 35.0% (166/474) of the videos were labeled as abnormal, and 43.0% (34/79) of CT studies were defined as abnormal.

The two cohorts were not significantly (P>0.622) different in terms of the percentages of the males, the percentage of abnormal videos, and the percentage of abnormal CT studies.

### Model Tuning Result

TABLE 1
Model Tuning Results. It shows that 4 systolic frames input
into a pre-trained Inception V3 CNN had highest accuracy.

| | | Pre-trained CNN for feature extraction | | | |
|---|---|---|---|---|---|
| | | Xception | InceptionResNetV2 | InceptionV3 | ResNet152V2 |
| N, number | 2 | 0.914 | 0.905 | 0.919 | 0.910 |
| of frames | 3 | 0.933 | 0.910 | 0.924 | 0.929 |
| | 4 | 0.924 | 0.919 | 0.938 | 0.933 |

Table 1 shows that all combinations of frames and pre-trained networks achieved high accuracy (>0.90). Inception V3 and N=4 frames achieved the highest per-video validation accuracy (=0.938). Therefore, we used this combination for as the final design.

### Model Performance

Per-video DL classification of WMA had high performance in cross-validation of training cases and in the testing cohort. For the cross-validation of training cases, accuracy=92.9%, sensitivity=90.9% and specificity=94.0%. This was similar for the testing cohort: accuracy=92.4%, sensitivity=89.2% and specificity=94.2%. Per-study DL classification was also high in both cohorts (training: accuracy=94.8%, sensitivity=93.1% and specificity=96.1%, testing: accuracy=97.5%, sensitivity=97.1% and specificity=97.8%).

TABLE 2
Confusion Matrices for Model Performance. In per-study classification, a CT study was labeled
as abnormal if >1 VR videos were abnormal. SE = sensitivity, SP = specificity, AC = accuracy.

| | | Training Cohort | | | | Testing Cohort | | | |
|---|---|---|---|---|---|---|---|---|---|
| | | Per-video | | Per-study | | Per-video | | Per-study | |
| | | Ground Truth | | Ground Truth | | Ground Truth | | Ground Truth | |
| | | Abnormal | Normal | Abnormal | Normal | Abnormal | Normal | Abnormal | Normal |
| DL | Abnormal | 331 | 41 | 67 | 4 | 148 | 18 | 33 | 1 |
| | Normal | 33 | 639 | 5 | 98 | 18 | 290 | 1 | 44 |
| | | SE | 0.909 | SE | 0.931 | SE | 0.892 | SE | 0.971 |
| | | SP | 0.940 | SP | 0.961 | SP | 0.942 | SP | 0.978 |
| | | AC | 0.929 | AC | 0.948 | AC | 0.924 | AC | 0.975 |

There were no statistically significant differences (P>0.34) between model performance in the training and testing cohorts. The confusion matrices are shown in Table 2.

### Data-size Reduction and Run Time

The average size of the CT study across one cardiac cycle was 1.52±0.67 Gigabytes. One VR video was 341±70 Kilobytes (2.00±0.40 Megabyte for 6 videos per study). Thus, VR videos led to a data size that is 778 times smaller than the conventional 4DCT study.

The framework was trained for 300 epochs in ˜0.5 hours in our workstation. It took 0.74±0.08 seconds to extract image features and predict WMA presence for all 6 videos of one CT study.

The disclosed technology can be implemented in some embodiments to provide a novel framework to efficiently (in terms of memory usage) represent wall motion and automatically detect WMA from 4DCT data with high accuracy. In some embodiments of the disclosed technology, volume rendering videos can significantly reduce the memory needs for cardiac CT functional assessment. In some embodiments of the disclosed technology, this volume rendering representation can be paired with a DL framework to accurately detect WMA. Both the VR representation and the classification of WMA can be performed automatically and quickly. More specifically, unlike current approaches which require complex high-dimensional computations involving point registration and motion field estimation, our framework predicts the presence of a WMA prediction in <1 second directly from 4 image frames obtained from the VR video. In addition, the disclosed technology can be implemented in some embodiments to analyze the complex 3D motion of the heart which may not be readily apparent using 2D approaches. The disclosed technology can be implemented in some embodiments to offer an automatic and very fast way to screen CT cases for WMA from highly compressed data, which may streamline the clinical pipeline.

In this way, WMA can be detected from the videos of the volume rendered LV endocardial blood-pool using a DL framework with high per-video and per-study accuracy.

The presence of cardiac (LV) wall motion abnormalities such as left ventricular (LV) wall motion abnormalities (WMA) is an independent indicator of adverse cardiovascular events in patients with cardiovascular diseases. We develop and evaluate the ability to detect cardiac wall motion abnormalities (WMA) from dynamic volume renderings (VR) of clinical 4D computed tomography (CT) angiograms using a deep learning (DL) framework.

In some example implementations, three hundred forty-three ECG-gated cardiac 4DCT studies (age: 61±15, 60.1% male) were retrospectively evaluated. Volume-rendering videos of the LV blood pool were generated from 6 different perspectives (i.e., six views corresponding to every 60-degree rotation around the LV long axis); resulting in 2058 unique videos. Ground-truth WMA classification for each video was performed by evaluating the extent of impaired regional shortening (measured in the original 4DCT data). DL classification of each video for the presence of WMA was performed by first extracting image features frame-by-frame using a pre-trained Inception network and then evaluating the set of features using a long short-term memory network. Data were split into 60% for 5-fold cross-validation and 40% for testing.

Volume rendering videos represent ˜800-fold data compression of the 4DCT volumes. Per-video DL classification performance was high for both cross-validation (accuracy=93.1%, sensitivity=90.0% and specificity=95.1%, κ: 0.86) and testing (90.9, 90.2, and 91.4% respectively, κ: 0.81). Per-study performance was also high (cross-validation: 93.7, 93.5, 93.8%, κ: 0.87; testing: 93.5, 91.9, 94.7%, κ: 0.87). By re-binning per-video results into the 6 regional views of the LV we showed DL was accurate (mean accuracy=93.1 and 90.9% for cross-validation and testing cohort, respectively) for every region. DL classification strongly agreed (accuracy=91.0%, κ: 0.81) with expert visual assessment.

Left Ventricular (LV) wall motion abnormalities (WMA) are an independent indicator of adverse cardiovascular events and death in patients with cardiovascular diseases such as myocardial infarction (MI), dyssynchrony and congenital heart disease. Further, regional WMA have greater prognostic values after acute MI than LV ejection fraction (EF). Multidetector computed tomography (CT) is routinely used to evaluate coronary arteries. Recently, ECG-gated acquisition of cardiac 4DCT enables the combined assessment of coronary anatomy and LV function. Recent publications show that regional WMA detection with CT agrees with echocardiography as well as with cardiac magnetic resonance.

Dynamic information of the 3D cardiac motion and regional WMA is encoded in 4DCT data. Visualization of regional WMA with CT usually requires reformatting the acquired 3D data along standard 2D short-and long-axis imaging planes. However, it requires experience in practice to resolve the precise region of 3D wall motion abnormalities from these 2D planes. Further, these 2D plane views may be confounded by through-plane motion and foreshortening artifacts. We propose to directly view 3D regions of wall motion abnormalities through the use of volumetric visualization techniques such as volume rendering (VR), which can preserve high resolution anatomical information and visualize 3D and 4D data simultaneously over large regions of the LV in cardiovascular CT. In VR, the 3D CT volume is projected onto a 2D viewing plane and different colors and opacities are assigned to each voxel based on intensity. It has been shown that VR provides a highly representative and memory efficient way to depict 3D tissue structures and anatomic abnormalities. The disclosed technology can be implemented in some embodiments to perform dynamic 4D volume rendering by sequentially combining the VR of each CT time frame into a video of LV function (we call this video a “Volume Rendering video”). The disclosed technology can be implemented in some embodiments to use volume rendering videos of 4DCT data to depict 3D motion dynamics and visualize highly local wall motion dynamics to detect regional WMA.

Analytical approaches to quantify 3D motion from 4DCT using image registration and deformable LV models have been developed. However, these approaches usually require complex and time-consuming steps such as user-guided image segmentation and point-to-point registration or feature tracking. Further, analysis of multiple frames at the native image resolution/size of 4DCT can lead to significant memory limitations, especially when running deep learning experiments using current graphical processing units (GPU). Volume rendering (VR) videos provide a high-resolution representation of 4DCT data which clearly depicts cardiac motion at a significantly reduced memory footprint (˜1 Gigabyte when using original 4DCT for motion analysis and only ˜100 kilobytes when using volume rendering video). Given the lack of methods currently available to analyze motion observed in VR videos, an objective observer can be created to automate VR video interpretation. Doing so would facilitate clinical adoption as it would avoid the need for training individuals on VR video interpretation and the approach could be readily shared. Deep learning approaches have been successfully used to perform classification of patients using medical images. Further, DL methods, once trained, are very inexpensive and can be easily deployed.

Therefore, the disclosed technology can be implemented in some embodiments to propose a novel framework which combines volume rendering videos of clinical cardiac CT cases with a DL classification to detect WMA. The disclosed technology can be implemented in some embodiments to provide a process to generate VR videos from 4DCT data and then to utilize a combination of a convolutional neural network (CNN) and recurrent neural network (RNN) to assess regional WMA observable in the videos.

### CT Data Collection

In some example implementations, 343 ECG-gated contrast enhanced cardiac CT patient studies between January 2018 and December 2020 were retrospectively collected. Inclusion criteria include: each study (a) had images reconstructed across the entire cardiac cycle, (b) had a field-of-view which captured the entire LV, (c) was free from significant pacing lead artifact in the LV and (d) had a radiology report including assessment of cardiac function. Images were collected by a single, wide detector CT scanner with 256 detector rows allowing for a single heartbeat axial 16 cm acquisition across the cardiac cycle. The CT studies were performed for range of clinical cardiac indications including suspected coronary artery disease (CAD, n=153), pre-procedure assessment of pulmonary vein isolation (PVI, n=126), preoperative assessment of transcatheter aortic valve replacement (TAVR, n=42), preoperative assessment of cardiac assist device placement (LVAD, n=22).

### Production of Volume Rendering Video of LV Blood-pool

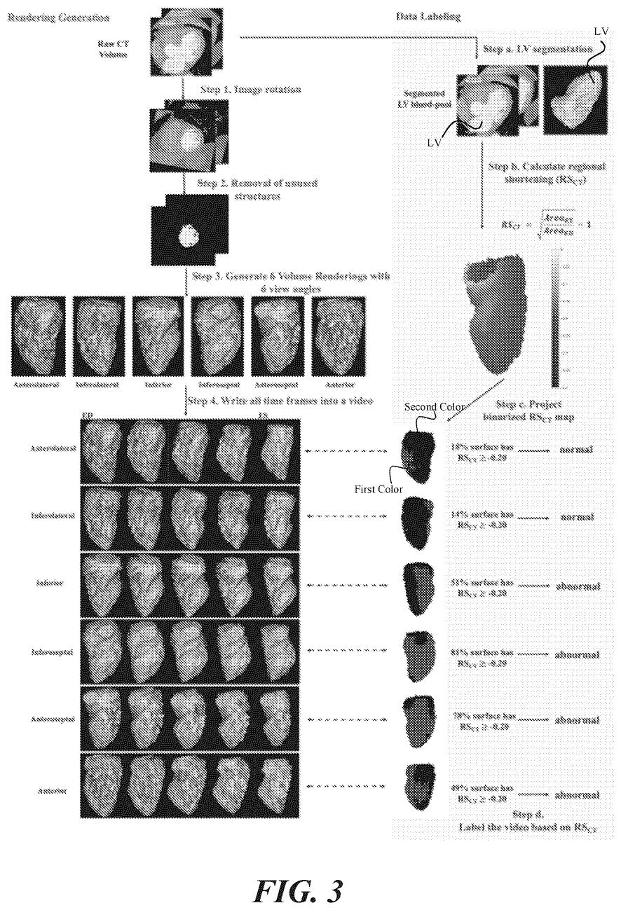

FIG. 3 shows automatic generation and quantitative labeling of volume rendering video based on some embodiments of the disclosed technology. Referring to FIG. 3, the disclosed technology can be implemented in some embodiments to include two operations: (1) rendering generation; and (2) data labeling. In some implementations, the rendering generation includes an automatic generation of VR video (left column, step 1-4). In some implementations, the data labeling includes quantitative labeling of the video (right column, step a-d).

In some implementations, the rendering generation includes, at steps 1 and 2, preparing the greyscale image of LV blood-pool with all other structures removed, at step 3, for each study, generating 6 volume renderings with 6 view angles rotated every 60 degrees around the long axis. The mid-cavity AHA segment in the foreground was noted under each view. In some implementations, the rendering generation includes, at step 4, for each view angle, creating a volume rendering video to show the wall motion across one heartbeat. Five systolic framesin VR video are presented. ED indicates end-diastole, and ES indicates end-systole.

In some implementations, the data labeling includes, at step a, LV segmentation, and at step b, calculating quantitative RS_{CT} for each voxel. In some implementations, at step c of the data labeling, the voxel-wise RS_{CT} map is binarized and projected onto the pixels in the VR video. See “Video Classification for the Presence of Wall Motion Abnormality” below. In rendered RS_{CT} map, the pixels with RS_{CT}≥−0.20 (abnormal wall motion) are labeled as a first color and those with RS_{CT}<−0.20 (normal) were labeled as a second color. In some implementations, the data labeling includes, at step d, labeling a video as abnormal if >35% endocardial surface has RS_{CT}≥−0.20 (first color pixels).

As shown in FIG. 3, steps 1-4 show the pipeline of VR video production. The CT images were first rotated using visual landmarks such as the RV insertion and LV apex, so that every study had the same orientation (with the LV long axis along the z-axis of the images and the LV anterior wall at 12 o′clock in cross-sectional planes). Structures other than LV blood-pool (such as LV myocardium, ribs, the right ventricle, and great vessels) were automatically removed by a pre-trained DL segmentation U-Net which has previously shown high accuracy in localizing the LV in CT images. If present, pacing leads were removed manually.

The resultant grayscale images of the LV blood-pool (as shown in FIG. 3 step 2) were then used to produce Volume renderings (VR) via MATLAB (version: 2019b, Math Works, Natick MA). Note the rendering was performed using the native CT scan resolution. The LV endocardial surface shown in VR was defined by automatically setting the intensity window level (WL) equal to the mean voxel intensity in a small ROI placed at the centroid of the LV blood pool and setting the window width (WW) equal to 150 HU (thus WL is study-specific, and WW is uniform for every study). Additional rendering parameters are listed in the section “Pre-set Parameters for Volume Rendering” below. VR of all frames spanning one cardiac cycle was then saved as a video (“VR video,” FIG. 3).

Each VR video projects the 3D LV volume from one specific projection view angle θ, thus it shows only part of the LV blood-pool and misses parts that are on the backside. Therefore, to see and evaluate all AHA segments, 6 VR videos were generated per study, with six different projection views θ_{60×n, n∈[0,1,2,3,4,5]} corresponding to 60-degree rotations around the LV long axis (see the section “Production of Six VR Videos for Each Study” below). With our design, each projection view had a particular mid-cavity AHA segment shown on the foreground (meaning this segment was the nearest to and in front of the ray source-point of rendering) as well as its corresponding basal and apical segments. Two adjacent mid-cavity AHA segments and their corresponding basal and apical segments were shown on the left and right boundary of the rendering in that view. In standard regional terminology, the six projection views (n=0, 1, 2, 3, 4, 5 in θ_{×n}) looked at the LV from the view with mid-cavity Anterolateral, Inferolateral,

Inferior, Inferoseptal, Anteroseptal and Anterior segments on the foreground, respectively. In this paper, to simplify the text we call them six “regional LV views” from anterolateral to anterior. In total, a large dataset of 2058 VR videos (343 patients×6 views) with unique projections were generated.

### Classification of Wall Motion

In FIG. 3, steps a-d show how the ground truth presence or absence of WMA at each location on the endocardium was determined. It is worth clarifying first that the ground truth is made on the original CT data not the volume rendered data. First, voxel-wise LV segmentations obtained using the U-Net were manually refined in ITK-SNAP (Philadelphia, PA, USA). Then, regional shortening (RS_{CT}) of the endocardium was measured using a previously-validated surface feature tracking technique. The accuracy of RS_{CT} in detecting WMA has been validated previously with strain measured by tagged MRI [a validated non-invasive approach for detecting wall motion abnormalities in myocardial ischemia]. Regional shortening can be calculated at each face on the endocardial mesh as:

$${RS}_{CT} = \sqrt{\frac{{Area}_{ES}}{{Area}_{ED}}} - 1$$

where Area_{ES} is the area of a local surface mesh at end-systole(ES) and Area_{ED} is the area of the same mesh at end-diastole (ED). ED and ES were determined based on the largest and smallest segmented LV blood-pool volumes, respectively. RS_{CT} for an endocardial surface voxel was calculated as the average RS_{CT} value of a patch of mesh faces directly connected with this voxel. RS_{CT} values were projected onto pixels in each VR video view (see the section “Video Classification for the Presence of Wall Motion Abnormality” below) to generate a ground truth map of endocardial function for each region from the perspective of each VR video. Then, each angular position was classified as abnormal (WMA present) if >35% of the endocardial surface in that view had impaired RS_{CT} (RS_{CT}−0.20). The section “Threshold Value Choices” below explains how these thresholds were selected.

To do per-study classification in this project, we defined that a CT study is abnormal if it has more than one VR videos labeled as abnormal (N_{ab_videos}≥2). Other thresholds (e.g., N_{ab_videos}≥1 or 3) were also chosen and the corresponding results were shown in the section “Per-study Classification with Different Threshold N_{ab_videos}” below.

### DL Framework Design

The DL framework (see FIG. 2) consists of three components, (a) a pre-trained 2D convolutional neural network (CNN) used to extract spatial features from each input frame of a VR video, (b) a recurrent neural network (RNN) designed to incorporate the temporal relationship between frames, and (c) a fully connected neural network designed to output the classification.

As shown in FIG. 2, an example of deep learning framework includes a plurality of components. Four frames were input into a pre-trained inception-v3 individually to obtain a 2048-length feature vector for each frame. Four vectors were concatenated into a feature matrix which was then input to the next components in the framework. A Long Short-term Memory followed by fully connected layers was trained to predict a binary classification of the presence of WMA in the video. CNN, convolutional neural network; RNN, recurrent neural network.

Given our focus on systolic function, four frames (ED, two systolic frames, and ES) were input to the DL architecture. This sampling was empirically found to maximize DL performance. Given the CT gantry rotation time, this also minimizes view sharing present in each image frame while providing a fuller picture of endocardial deformation. Each frame was resampled to 299×299 pixels to accommodate the input size of the pre-trained CNN.

Component (a) is a pre-trained CNN with the Inception architecture (Inception-v3) and the weights obtained after training on the ImageNet database. The reason to pick Inception-v3 architecture can be found in this reference. This component was used to extract features and create a 2048-length feature vector for each input image. Feature vectors from the four frames were then concatenated into a 2D feature matrix with size=(4, 2048).

Component (b) is a long short-term memory RNN with 2048 nodes, tanh activation and sigmoid recurrent activation. This RNN analyzed the (4, 2048) feature matrix from component (a) to synthesize temporal information (RNN does this by passing the knowledge learned from the previous instance in a sequence to the learning process of the current instance in that sequence then to the next instance). The final component (c), the fully connected layer, logistically regressed the binary prediction of the presence of WMA in the video.

### Cross-Validation and Testing

In our DL framework, component (a) was pre-trained and directly used for feature extraction whereas components (b) and (c) were trained end-to-end as one network for WMA classification. Parameters were initialized randomly. The loss function was categorical cross-entropy.

The dataset was split randomly into 60% and 40% subsets. 60% (205 studies, 1230 videos) were used for 5-fold cross-validation, meaning in each fold of validation we had 164 studies (984 videos) to train the model and the rest 41 studies (246 videos) to validate the model. We report model performance across all folds. 40% (138 studies, 828 videos) were used only for testing.

### Experiment Settings

We performed all DL experiments using TensorFlow on a workstation. The file size of each 4DCT study and VR video were recorded. Further, the time needed to run each step in the entire framework (including the image processing, VR video generation and DL prediction) on the new cases was recorded.

### Model Performance and Left Ventricular Ejection Fraction (LVEF)

The impact of systolic function, measured via LVEF on DL classification accuracy was evaluated in studies with LVEF<40%, LVEF between 40-60%, LVEF>60%. We hypothesized that the accuracy of the model would be different for different LVEF intervals since because the “obviously abnormal” LV with low EF, and the “obviously normal” LV with high EF would be easier to classify. The consequence of a local WMA in hearts with LVEF between 40-60% might be a more subtle pattern and harder to detect. These subtle cases are also difficult for human observers.

Comparison with Expert Visual Assessment

While not the primary goal of the study we investigated the consistency of the DL classifications with the results from two human observers using traditional views. 100 CT studies were randomly selected from the testing cohort for independent analysis of WMA by two cardiovascular imaging experts with different levels of experiences: expert 1 with >20 years of experience and expert 2 with >5 years of experience. The experts classified the wall motion in each AHA segment into 4 classes (normal, hypokinetic, akinetic and dyskinetic) by visualizing wall motion from standard 2D short-and long-axis imaging planes, in a blinded fashion. Because of the high variability in the inter-observer classifications of abnormal categories, the disclosed technology can be implemented in some embodiments to (1) combine the last three classes into a single “abnormal” class indicating WMA detection, and (2) perform the comparison on a per-study basis. A CT study was classified as abnormal by the experts if it had more than one abnormal segment. The interobserver variability is reported in the result Section Model performance-comparison with expert assessment. It should be noted that our model was only trained on ground truth based on quantitative RS_{CT} values; the expert readings were performed as a measure of consistency with clinical performance.

### Statistical Evaluation

Two-tailed categorical z-test was used to compare data proportions (e.g., proportions of abnormal videos) in two independent cohorts: a cross-validation cohort and a testing cohort. Statistical significance was set at P≤0.05.

DL Model performance against the ground truth label was reported via confusion matrix and Cohen's kappa value. Both regional (per-video) and per-study comparison were performed. A CT study is defined as abnormal if it has more than one VR videos labeled as abnormal (N_{ab_videos}≥2). As stated in Section Production of volume rendering video of LV blood-pool, every projection view of the VR video corresponded to a specific regional LV view. Therefore, we re-binned the per-video results into 6 LV views to test the accuracy of the DL model when looking at each region of the LV. We also calculated the DL per-study accuracy for patients with each clinical cardiac indication in the testing cohort and use pair-wise Chi-squared test to compare the accuracies between indications.

### Results

Of the 1230 views (from 205 CT studies) used for 5-fold cross-validation, 732 (from 122 studies, 59.5%) were male (age: 63±15) and 498 (from 83 studies, 40.5%) were female (age: 62±15). The LV blood pool had a median intensity of 516 HU (IQR: 433 to 604). 40.0% (492/1230) of the videos were labeled as abnormal based on RS_{CT} analysis, and 45.4% (93/205) of studies had WMA in ≥2 videos. 104 studies had LVEF >60%, 54 studies had LVEF <40% and the rest 47 (47/205=22.9%) studies had LVEF between 40-60%. For clinical cardiac indications, 85 studies have suspect CAD, 77 studies have the pre-PVI assessment, 31 studies have the pre-TAVR assessment, and 12 studies have the pre-VAD assessment.

Of the 828 views (from 138 CT studies) used for testing, 504 (from 84 studies, 60.9%) were male (age: 57±16) and 324 (from 54 studies, 39.1%) were female (age: 63±13). The LV blood pool had a median intensity of 520 HU (IQR: 442 to 629). 37.0% (306/828) of the videos were labeled as abnormal, and 45.0% (62/138) of studies had WMA in ≥2 videos. 72 studies had LVEF >60%, 25 studies had LVEF <40% and the rest 41 (41/138=28.7%) studies had LVEF between 40-60%. For clinical cardiac indications, 68 studies have suspect CAD, 49 studies have the pre-PVI assessment, 11 studies have the pre-TAVR assessment, and 10 studies have the pre-VAD assessment.

There were no significant differences (all P-values>0.05) in data proportions between the cross-validation and testing cohorts in terms of the percentages of sex, abnormal videos, abnormal CT studies.

### Model Performance—Per-Video and Per-Study Classification

TABLE 3
DL classification performance in cross-validation and testing

| | | Cross-validation | | | | Testing | | | |
|---|---|---|---|---|---|---|---|---|---|
| | | | | Per-study | | | | Per-study | |
| | | Per-video | | (N_{ab}_{_{—}}_{videos} ≥ 2) | | Per-video | | (N_{ab}_{_{—}}_{videos} ≥ 2) | |
| | | Ground truth | | Ground truth | | Ground truth | | Ground truth | |
| | | Abnormal | Normal | Abnormal | Normal | Abnormal | Normal | Abnormal | Normal |
| DL | Abnormal | 443 | 36 | 87 | 7 | 276 | 45 | 57 | 4 |
| | Normal | 49 | 702 | 6 | 105 | 30 | 477 | 5 | 72 |
| | | Sens | 0.900 | Sens | 0.935 | Sens | 0.902 | Sens | 0.919 |
| | | Spec | 0.951 | Spec | 0.938 | Spec | 0.914 | Spec | 0.947 |
| | | Acc | 0.931 | Acc | 0.937 | Acc | 0.909 | Acc | 0.935 |
| | | κ | 0.855 | κ | 0.872 | κ | 0.808 | κ | 0.868 |

Per-video and per-study DL classification performance for WMA were excellent in both cross-fold validation and testing. Table 3 shows that the per-video classification for the cross-validation had high accuracy=93.1%, sensitivity=90.0% and specificity=95.1%, Cohen's kappa κ=0.86 with 95% CI as [0.83, 0.89]. Per-study classification also had excellent performance with accuracy=93.7%, sensitivity=93.5% and specificity=93.8%, κ=0.87[0.81, 0.94]. Table 3 also shows that the per-video classification for the testing cohort had high accuracy=90.9%, sensitivity=90.2% and specificity=91.4%, κ=0.81[0.77, 0.85]. We obtained per-study classification accuracy=93.5%, sensitivity=91.9% and specificity=94.7%, κ=0.87[0.78, 0.95] in the testing cohort.

Two hundred five CT studies and 1230 Volume Rendered (VR) videos were used for 5-fold cross-validation. One hundred thirty-eight CT studies and 828 VR videos were in the testing. The four confusion matrices correspond to per-video classification and per-study classification for cross-validation (left) and testing (right). N_{ab_videos}>2 (number of views classified as abnormal) was used to classify a study as abnormal. Sens, sensitivity; Spec, specificity; Acc, accuracy. Cohen's kappa κ is also reported.

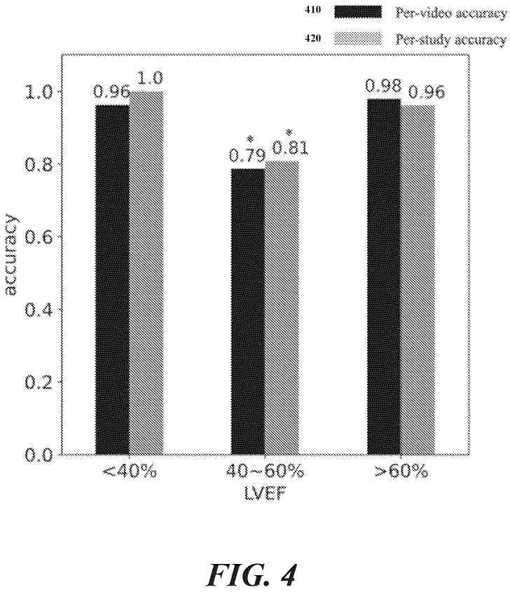

FIG. 4 shows the relationship between DL classification accuracy and LVEF in the cross-validation. The per-video (410) and per-study (420) accuracy are shown in studies with (LVEF<40%), (40<LVEF<60%) and (LVEF>60%) (“*” indicates the significant difference).

TABLE 4
DL classification performance in CT studies with 40 < LVEF < 60%.

| | | Cross-validation | | | | Testing | | | |
|---|---|---|---|---|---|---|---|---|---|
| | | | | Per-study | | | | Per-study | |
| | | Per-video | | (N_{ab}_{_{—}}_{videos} ≥ 2) | | Per-video | | (N_{ab}_{_{—}}_{videos} ≥ 2) | |
| | | Ground truth | | Ground truth | | Ground truth | | Ground truth | |
| | | Abnormal | Normal | Abnormal | Normal | Abnormal | Normal | Abnormal | Normal |
| DL | Abnormal | 131 | 23 | 33 | 5 | 126 | 23 | 32 | 3 |
| | Normal | 37 | 91 | 4 | 5 | 26 | 71 | 1 | 5 |
| | | Sens | 0.780 | Sens | 0.892 | Sens | 0.829 | Sens | 0.970 |
| | | Spec | 0.798 | Spec | 0.500 | Spec | 0.755 | Spec | 0.625 |
| | | Acc | 0.787 | Acc | 0.809 | Acc | 0.801 | Acc | 0.902 |
| | | κ | 0.567 | κ | 0.407 | κ | 0.581 | κ | 0.657 |

Table 4 shows that CT studies with LVEF between 40 and 60% in the cross-validation cohort were classified with per-video accuracy=78.7%, sensitivity=78.0% and specificity=79.8%. In the testing cohort, per-video classification accuracy=80.1%, sensitivity=82.9% and specificity=75.5% accuracy for this LVEF group remained relatively high but was lower (P<0.05) than the accuracy for patients with LVEF<40% and LVEF>60% due to the more difficult nature of the classification task in this group with more “subtle” wall motion abnormalities.

Forty-seven CT studies with 40%<LVEF<60% were in the cross-validation and 41 CT studies were in the testing.

### Model Performance—Regional LV Views

TABLE 5
Results re-binned into six regional LV views.

| | | Per-video classification | | | | | | | |
|---|---|---|---|---|---|---|---|---|---|
| Projection | LV wall on the | Cross-validation | | | | Testing | | | |
| view | foreground | Sens | Spec | Acc | κ | Sens | Spec | Acc | κ |
| 0 | Anterolateral | 0.845 | 0.964 | 0.922 | 0.824 | 0.886 | 0.936 | 0.920 | 0.818 |
| 60 | Inferolateral | 0.938 | 0.952 | 0.946 | 0.888 | 0.909 | 0.915 | 0.913 | 0.805 |
| 120 | Inferior | 0.879 | 0.974 | 0.932 | 0.860 | 0.917 | 0.910 | 0.913 | 0.824 |
| 180 | Inferoseptal | 0.882 | 0.946 | 0.917 | 0.832 | 0.847 | 0.861 | 0.855 | 0.705 |
| 240 | Anteroseptal | 0.963 | 0.944 | 0.951 | 0.899 | 0.927 | 0.952 | 0.942 | 0.879 |
| 300 | Anterior | 0.893 | 0.931 | 0.917 | 0.822 | 0.932 | 0.904 | 0.913 | 0.807 |

Table 5 shows that our DL model was accurate for detection of WMA in all 6 regional LV views both in cross-validation cohort (mean accuracy=93.1%±0.03) and testing cohort (mean accuracy=90.9%±0.06).

This table shows the per-video classification of our DL model when detecting WMA from each regional view of LV. See the definition of regional LV views in Section Production of volume rendering video of LV blood-pool. Sens, sensitivity; Spec, specificity; Acc, accuracy.

### Model Performance—Different Clinical Cardiac Indications

We calculated the DL per-study classification accuracy equal to 91.2% for CT studies with suspect CAD (n=68 in the testing cohort), 93.9% for studies with pre-PVI assessment (n=49), 100% for patients with pre-TAVR assessment (n=11), 100% for studies with pre-LVAD assessment (n=10). Using Chi-squared test pairwise, there was no significant difference of DL performance between indications (all P-values>0.5).

Model Performance—Comparison with Expert Assessment

First, we report the interobserver variability of two experts. The Cohen's kappa for the agreement between observers on per-AHA-segment basis was 0.81[0.79, 0.83] and on the per-CT-study basis was 0.88[0.83, 0.93]. For those segments labeled as abnormal by both experts, the Kappa for the two experts to further classify an abnormal segment into hypokinetic, akinetic and dyskinetic dramatically dropped to 0.34.

Second, we show in the Table 6 that per-study comparison between DL prediction and expert visual assessment on 100 CT studies in the testing cohort led to Cohen's Kappa κ=0.81[0.70,0.93] for expert 1 and κ=0.73[0.59,0.87] for expert 2.

TABLE 6
Comparison between DL and expert visual assessment.

| | | Expert visual assessment | | | |
|---|---|---|---|---|---|
| | | Expert 1 | | Expert 2 | |
| | | Abnormal | Normal | Abnormal | Normal |
| DL | Abnormal | 37 | 5 | 33 | 9 |
| | Normal | 4 | 54 | 4 | 54 |
| | | κ | 0.815 | κ | 0.729 |

Per-study comparison were run on 100 CT studies randomly selected from the testing cohort. The columns “Expert 1” indicate per-video evaluation, and the columns “Expert 2” indicate per-study evaluation.

### Data-Size Reduction

The average size of the CT study across one cardiac cycle was 1.52±0.67 Gigabytes. One VR video was 341±70 Kilobytes, resulting in 2.00±0.40 Megabytes for 6 videos per study. VR videos led to a data size that is ˜800 times smaller than the conventional 4DCT study.

### Run Time

Regarding image processing, the image rotation took 14.1±1.2 seconds to manually identify the landmarks and then took 38.0±16.2 seconds to automatically rotate the image using the direction vectors derived from landmarks. The DL automatic removal of unnecessary structures took 141.0±20.3 seconds per 4DCT study. If needed, manual pacing lead artifacts removal took around 5-10 mins per 4DCT study depending on the severity of artifacts. Regarding automatic VR video generation, it took 32.1±7.0 seconds (to create 6 VR videos from the processed CT images). Regarding DL prediction of WMA presence in one CT study, it took 0.7±0.1 seconds to extract image features from frames of the video and took ˜0.1 seconds to predict binary classification for all 6 VR videos in the study. To summarize, the entire framework requires approximately 4 minutes to evaluate a new study if no manual artifacts removal is needed.

The disclosed technology can be implemented in some embodiments to provide a DL framework that detects the presence of WMA in dynamic 4D volume rendering (VR videos) depicting the motion of the LV endocardial boundary. VR videos enabled a highly compressed (in terms of memory usage) representation of large regional fields of view with preserved high spatial-resolution features in clinical 4DCT data. Our framework analyzed four frames spanning systole extracted from the VR video and achieved high per-video (regional LV view) and per-study accuracy, sensitivity and specificity (≥0.90) and concordance (κ≥0.8) both in cross-validation and testing.

### Benefits of the Volume Visualization Approach

Assessment of regional WMA with CT is usually performed on 2D imaging planes reformatted from the 3D volume. However, 2D approaches often confuse the longitudinal bulk displacement of tissue into and out of the short-axis plane with true myocardial contraction. Various 3D analytical approaches to quantify 3D motion using image registration and deformable LV models have been developed; our novel use of regional VR videos as input to DL networks has several benefits when compared to these traditional methods. First, VR videos contain 3D endocardial surface motion features which are visually apparent. This enables simultaneous observation of the complex 3D motion of a large region of the LV in a single VR video instead of requiring synthesis of multiple 2D slices. Second, our framework is extremely memory efficient with reduced data size while preserving key anatomical and motion information; a set of 6 VR videos is ˜800 times smaller in data size than the original 4DCT data. The use of VR videos also allows our DL experiments to run on the current graphic processing unit (GPU), whereas the original 4DCT data is too large to be imported into the GPU. Third, our framework is simple as it does not require complex and time-consuming computations such as point registration or motion field estimation included in analytical approaches. The efficiency of our technique will enable retrospective analysis of large numbers of functional cardiac CT studies; this cannot be said for traditional 3D tracking methods which require significant resources and time for segmentation and analysis.

### Model Performance for Each LV View

We re-binned the per-video results into 6 projection views corresponding to 6 regional LV views and showed that our DL model is accurate to detect WMA from specific regions of the LV. The results shown in table above indicate that all results for classification can be labeled with a particular LV region. For example, to evaluate the wall motion on the inferior wall of a CT study, the classification from the VR video with the corresponding projection view θ(=120) would be used.

Comparison with Experts and Its Limitations

To evaluate the consistency of our model with standard clinical evaluation, we compared DL results with two cardiovascular imaging experts and showed high per-study classification correspondence. This comparison study has its limitations. First, we did not perform a per-AHA-segment comparison. Expert visual assessment was subjective (by definition) and had greater inter-observer variability on per-AHA-segment basis than the per-study basis the variability (Kappa increased from 0.81 for per-segment to 0.88 for per-study). Second, the interobserver agreement for experts to further classify an abnormal motion as hypokinetic, akinetic or dyskinetic was also too poor (Kappa=0.34) to use expert visual labels for three severities as the ground truth; therefore, we used one “abnormal” class instead of three levels of severity of WMA. Third, experts could only visualize the wall motion from 2D imaging planes while our DL model evaluated the 3D wall motion from VR videos. A future study using a larger number of observers, and a larger number of cases could be performed in which trends could be observed; however, it is clear that variability in subjective calls for degree of WMA will likely persist in the expert readers.

### Using RS_{CT} for Ground Truth Labeling

Direct visualization of wall motion abnormalities in volume rendered movies from 4DCT is a truly original application; hence, as can be expected there are no current clinical standards/guidelines for visual detection of WMA from volume rendered movies. In fact, we believe our paper is the first to introduce this method of evaluating myocardial function in a formal pipeline. In our recent experience, visual detection of patches of endocardial “stasis” in these 3D movies highly correlates with traditional markers of WMA such as wall thickening, circumferential shortening and longitudinal shortening. However, specific guidance on how to clinically interpret VR movies is not yet available. We expect human interpretation to depend on both experience and training. Thus, we used quantitative regional myocardial shortening (RSCT) derived from segmentation and 3D tracking to delineate regions of endocardial WMA. RS_{CT} has been previously shown to be a robust method for quantifying regional LV function.

### Limitations and Future Directions

First, our current DL pipeline has several manual image processing such as manual rotation of the image and manual removal of lead artifacts. These steps lengthen the time required to run the entire pipeline (see Section Run time) and limit the clinical utility. One important future direction of our technique is to integrate the DL-driven automatic image processing to get a fully automatic pipeline. Chen et al. have proposed a DL technique to define the short-axis planes from CT images so that the LV axis can be subsequently derived for correct image orientation. Zhang and Yu and Ghani and Karl have proposed DL techniques to remove the lead artifacts.

Second, our work only focuses on the systolic function and only takes 4 systolic frames from the VR video as the model input. The future direction is to input diastolic frames into the model to enable the evaluation of diastolic function and to use a 4D spatial-temporal convolutional neural network to directly process the video without requiring explicit selection of temporal frames.

Third, we currently perform binary classification of the presence of WMA in the video. The DL model integrates all information from all the AHA segments that can be seen in the video and only evaluates the extent of pixels with WMA (i.e., whether it's larger than 35% of the total pixels). The DL evaluation is independent of the position of WMA; thus, we do not identify which of the AHA segments contribute to the WMA just based on the DL binary classification. Future research is needed to “focus” the DL model's evaluation on specific AHA segments using such as local attention and evaluate whether the approach can delineate the location and extent of WMA in terms of AHA segments. Further, by using a larger dataset with a balanced distribution of all four severities of WMA, we aim to train the model to estimate the severity of the WMA in the future.

Fourth, tuning the inception V3 (the CNN) weights to extract features most relevant to detection of WMA is expected to further increase performance as it would further optimize how the images are analyzed. However, given our limited training data, we chose not to train weights of the inception network and the high performance we observed seems to have supported this choice.

In this way, the disclosed technology can be implemented in some embodiments to combine the video of the volume rendered LV endocardial blood pool with deep learning classification to detect WMA and observed high per-region (per-video) and per-study accuracy. This approach has promising clinical utility to screen for cases with WMA simply and accurately from highly compressed data.

### 1. Volume Rendering in MATLAB

### A. Pre-set Parameters for Volume Rendering

A built-in volume rendering function in MATLAB called “volshow” was used to automatically generate VR from 3D CT volume. Since in the preprocessing every CT volume was rotated to have a uniform orientation, a same set of camera-related parameters could be used across the entire dataset: “CameraPosition” was [6,0,1], “CameraUp Vector” was [0,0,1],“Camera ViewAngle” was 15°. CT image was normalized based on the study-specific window level and window width. See section “automated volume rendering video generation” in the main text for how to set these window level and window width. The built-in colormap (“hot”) and a linear alphamap was applied to the normalized CT image, assigning colors and opacities to each voxel according to its intensity. The background color was set to be black, and the lighting effect was turned on.

### B. Production of Six VR Videos for Each Study

Each VR video shows the projection of the 3D CT volume at one specific view angle θ. To evaluate all AHA segments, 6 VR videos, with six different views θ_{6×n, n∈[0,1,2,3,4,5]} corresponding to 60-degree clockwise rotations around the LV long axis, were generated for each study. The rotation of the camera was done automatically by applying a rotation matrix to the parameter “CameraPosition” for each video. The rotation was around the LV long axis which is the z-axis of the image, so the “CameraPosition” for a video with view angle θ can be calculated as:

$$[ \begin{matrix}px \\ py \\ pz\end{matrix} ] = [ \begin{matrix}cos ( θ ) & - sin ( θ ) & 0 \\ sin ( θ ) & cos ( θ ) & 0 \\ 0 & 0 & 1\end{matrix} ] \times [ \begin{matrix}6 \\ 0 \\ 1\end{matrix} ]$$

where [6 0 1] is the pre-set “CameraPosition” and [px py pz] is the derived “CameraPosition” for each view angle. All other rendering parameters were kept constant for every video.

### 2. Video Classification for the Presence of Wall Motion Abnormality

This section explains how we classified the WMA presence in a VR video with view angle θ based on the per-voxel RS_{CT} map for the endocardium.

The pipeline follows the steps below:

Step 1. Binarize the per-voxel RS_{CT} map using a threshold RS_{CT}*.

Step 2. Use the MATLAB built-in function “labelvolshow” to get the rendering image R_{RS} of the binary RS_{CT} map with the same view angle θ of the VR video (see an example of labeled rendering R_{RS} in FIG. 3 step c). “Labelvolshow” is a function to display the rendering of labeled volumetric data. All camera-related rendering parameters were kept the same as those for the VR video. As a result, R_{RS} displays the same endocardial surface as the VR video does.

Step 3. Count the number of abnormal pixels in R_{RS} and calculate its

$$percentage = \frac{{n}_{abnormal pixels}}{{n}_{abnormal pixels} + {n}_{normal pixels}} \times 100 \% .$$

A VR video is labeled as abnormal if >35% of the pixels in the R_{RS} (equivalently, >35% of the endocardial surface of LV) are abnormal.

In conclusion, a VR video was classified as abnormal (presence of WMA) if >35% of the endocardial surface of the LV had RS_{CT}≥RS_{CT}*. Here we set RS_{CT}*=−0.20.

### 2. A. Threshold Value Choices

A VR video is classified as abnormal if >35%. Here >35% was set based on the following derivation: since each projected view showed 3 AHA walls, if one AHA wall has WMA then approximately one-third (≈35%) of the projected CT would have abnormal RS_{CT}.

The threshold RS_{CT}*≥−0.20 was set based on the previous research. They showed the average RS_{CT} for a cohort of 23 healthy controls is equal to −0.32±0.06. Our threshold number RS_{CT}* to detect abnormal regions (WMA) was two standard deviations above the mean in normal cases=−0.20.

3. Per-study Classification with Different Threshold N_{ab_videos}

TABLE 7
per-study classification when a study is defined as abnormal with at least
one VR video labeled as abnormal (N_{ab}_{_{—}}_{videos} ≥ 1)

| | | Cross-Validation | | Testing | |
|---|---|---|---|---|---|
| | | Ground Truth | | Ground Truth | |
| | | Abnormal | Normal | Abnormal | Normal |
| DL | Abnormal | 92 | 9 | 62 | 14 |
| | Normal | 4 | 100 | 4 | 58 |
| | | Sens | 0.958 | Sens | 0.939 |
| | | Spec | 0.917 | Spec | 0.806 |
| | | Acc | 0.937 | Acc | 0.870 |
| | | κ | 0.873 | κ | 0.740 |

TABLE 8
per-study classification when a study is defined as abnormal with more
than two VR video labeled as abnormal (N_{ab}_{_{—}}_{videos} ≥ 3)

| | | Cross-Validation | | Testing | |
|---|---|---|---|---|---|
| | | Ground Truth | | Ground Truth | |
| | | Abnormal | Normal | Abnormal | Normal |
| DL | Abnormal | 81 | 5 | 52 | 1 |
| | Normal | 6 | 113 | 5 | 80 |
| | | Sens | 0.931 | Sens | 0.912 |
| | | Spec | 0.958 | Spec | 0.988 |
| | | Acc | 0.946 | Acc | 0.957 |
| | | κ | 0.890 | κ | 0.909 |

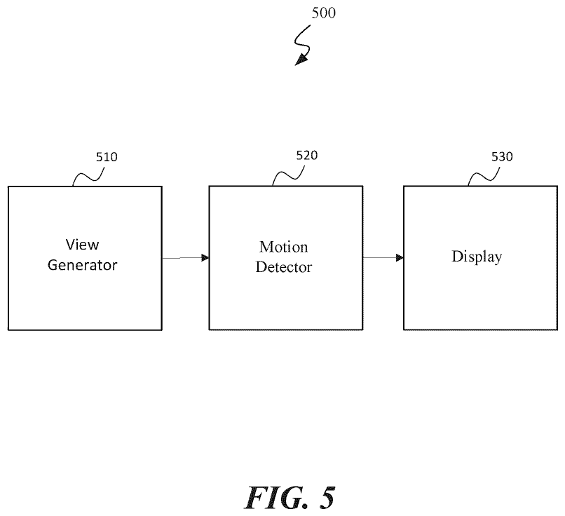

FIG. 5 shows an example system 500 implemented based on some embodiments of the disclosed technology.

In some implementations, the system 500 may include a view generator 510 configured to create a plurality of volume rendered views of an organ of a patient, a motion detector 520 coupled to the view generator to detect a regional motion of a section of the organ based on the plurality of volume rendered views of the organ, and a display 530 coupled to the motion detector to show the plurality of volume rendered views or a detection of an abnormality of the section using an image processing algorithm.

In some implementations, the view generator 510 may be configured to receive a medical image of a patient as an input and create a view of the medical image in accordance with a set of viewing parameters such as color codes, contrast and brightness levels, and zoom levels, for example. In some implementations, the view generator 510 may include one or more processors to read executable instructions to create volume rendered views out of, for example, computed tomography (CT) scans or magnetic resonance imaging (MRI) scans.

In some implementations, the motion detector 520 may include one or more processors to read executable instructions to detect a regional motion of a section of the organ based on the plurality of volume rendered views of the organ. In some implementations, the motion detector 520 may include one or more neural networks to detect and classify a severity of a motion abnormality of the organ. In some implementations, the motion detector 520 may include a first network to extract spatial features from each input frame of the plurality of volume rendered views of the organ; a second network to extract temporal information from a sequence of volume rendered frames corresponding to the plurality of volume rendered views of the organ; and an algorithm to classify a severity of a motion abnormality of the organ.

In some implementations, the display 530 may be configured to show the severity of the motion abnormality of the organ by assigning different colors to different levels of the severity of the motion abnormality of the organ.

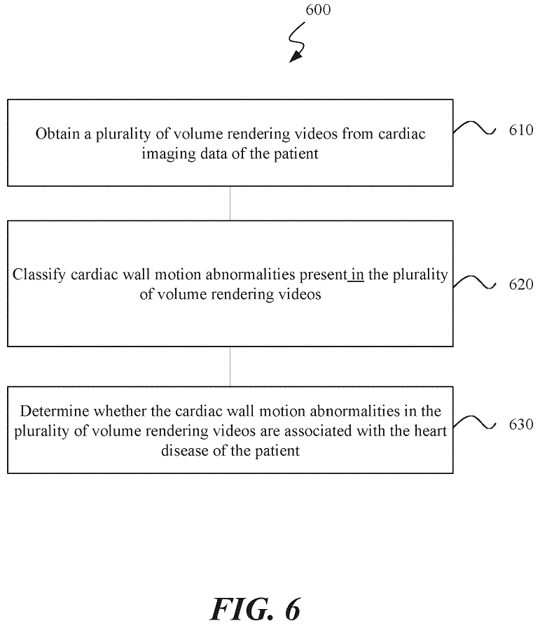

FIG. 6 is a flow diagram that illustrates an example method 600 for detecting a heart disease of a patient based on some embodiments of the disclosed technology. In some implementations, the method 600 may include, at 610, obtaining a plurality of volume rendering videos from cardiac imaging data of the patient, at 620, classifying cardiac wall motion abnormalities present in the plurality of volume rendering videos, and at 630, determining whether the cardiac wall motion abnormalities in the volume rendering videos are associated with the heart disease of the patient.

Therefore, various implementations of features of the disclosed technology can be made based on the above disclosure, including the examples listed below.

Example 1. A system, comprising: a view generator to create a plurality of volume rendered views of an organ of a patient; a motion detector coupled to the view generator to detect a regional motion of a section of the organ based on the plurality of volume rendered views of the organ; and a display coupled to the motion detector to show the plurality of volume rendered views or a detection of an abnormality of the section.

Example 2. The system of example 1, wherein the section of the organ includes a heart chamber of the patient.

Example 3. The system of example 1, wherein the regional motion of the section of the organ includes a myocardial wall motion of the patient.

Example 4. The system of example 1, wherein the abnormality includes a regional ischemia or infarction.

Example 5. The system of example 1, wherein the abnormality includes a change in a cardiac (LV) function.

Example 6. The system of example 1, wherein the plurality of volume rendered views includes at least one of size, shape, or border zone of an infarct.

Example 7. The system of example 1, wherein the motion detector is configured to include a deep learning network.

Example 8. The system of example 7, wherein the deep learning network includes: a first network to extract spatial features from each input frame of the plurality of volume rendered views of the organ; a second network to extract temporal information from a sequence of volume rendered frames corresponding to the plurality of volume rendered views of the organ; and an algorithm to classify a severity of a motion abnormality of the organ.

Example 9. The system of example 8, wherein the first network includes a pre-trained convolutional neural network (CNN), and the second network includes a recurrent neural network (RNN).

Example 10. A system comprising: a view generator to create a plurality of volume rendered views of an organ of a patient; a motion detector coupled to the view generator and including: a first network to extract spatial features from each input frame of the plurality of volume rendered views of the organ; a second network to extract temporal information from a sequence of volume rendered frames corresponding to the plurality of volume rendered views of the organ; and an algorithm to classify a severity of a motion abnormality of the organ; and a display coupled to the motion detector to show the severity of the motion abnormality of the organ by assigning different colors to different levels of the severity of the motion abnormality of the organ.

Example 11. The system of example 10, wherein the plurality of volume rendered views of the organ includes a view showing a myocardial wall motion of the patient.

Example 12. The system of example 10, wherein the motion abnormality of the organ includes a regional ischemia or infarction.

Example 13. The system of example 10, wherein the motion abnormality of the organ includes a change in a cardiac (LV) function.

Example 14. The system of example 10, wherein the plurality of volume rendered views includes at least one of size, shape, or border zone of an infarct.

Example 15. A method for detecting heart disease in a patient, comprising: obtaining a plurality of volume rendering videos from cardiac imaging data of the patient; classifying cardiac wall motion abnormalities present in the plurality of volume rendering videos; and determining whether the cardiac wall motion abnormalities in the plurality of volume rendering videos are associated with the heart disease of the patient.

In some implementations, classifying the cardiac wall motion abnormalities present in the plurality of volume rendering videos includes: determining regional shortenings (RS) of an endocardial surface between end-diastole and end-systole; and determining whether an area of the endocardial surface having the regional shortenings exceeds a threshold value.

In some implementations, determining whether the cardiac wall motion abnormalities in the volume rendering videos are associated with the heart disease of the patient includes: classifying the endocardial surface as abnormal upon determining that the area of the endocardial surface having the regional shortenings exceeds the threshold value.

Example 16. The method of example 15, wherein the cardiac imaging data includes cardiac computed tomography (CT) data.

Example 17. The method of example 15, wherein the cardiac wall motion abnormalities include left ventricular (LV) wall motion abnormalities.

Example 18. The method of example 15, wherein determining whether the cardiac wall motion abnormalities in the volume rendering videos are associated with the heart disease of the patient includes: extracting spatial features from each of input frames of the plurality of volume rendering videos; synthesizing a temporal relationship between the input frames; and generating a classification based on the extracted spatial features and the synthesized temporal relationship.

Example 19. The method of example 18, wherein the spatial features are extracted using a pre-trained convolutional neural network (CNN) configured to create N length feature vectors for each of the input frames, wherein Nis a positive integer.

Example 20. The method of example 19, wherein the temporal relationship between the input frames is synthesized using a recurrent neural network (RNN) configured to include a long short-term memory architecture with N nodes and a sigmoidal activation function.

Example 21. The method of example 20, wherein the RNN is configured to receive a feature sequence from the CNN and incorporate the temporal relationship.

Example 22. The method of example 18, wherein the classification is generated using a fully connected neural network.

Example 23. The method of example 18, wherein the fully connected neural network is configured to estimate a severity of cardiac wall motion abnormalities in the plurality of volume rendering videos.

Example 24. A system for detecting a heart disease of a patient, comprising a memory and a processor, wherein the processor reads code from the memory and implements a method recited in any of examples 16-23.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A system, comprising:
a view generator to create a plurality of volume rendered views of an organ of a patient;
a motion detector coupled to the view generator to detect a regional motion of a section of the organ based on the plurality of volume rendered views of the organ; and
a display coupled to the motion detector to show the plurality of volume rendered views or a detection of an abnormality of the section.

2. The system of claim 1, wherein the section of the organ includes a heart chamber of the patient.

3. The system of claim 1, wherein the regional motion of the section of the organ includes a myocardial wall motion of the patient.

4. The system of claim 1, wherein the abnormality includes a regional ischemia or infarction.

5. The system of claim 1, wherein the abnormality includes a change in a cardiac (LV) function.

6. The system of claim 1, wherein the plurality of volume rendered views includes at least one of size, shape, or border zone of an infarct.

7. The system of claim 1, wherein the motion detector is configured to include a deep learning network.

8. The system of claim 7, wherein the deep learning network includes:
a first network to extract spatial features from each input frame of the plurality of volume rendered views of the organ;
a second network to extract temporal information from a sequence of volume rendered frames corresponding to the plurality of volume rendered views of the organ; and
an algorithm to classify a severity of a motion abnormality of the organ, wherein the first network includes a pre-trained convolutional neural network (CNN), and the second network includes a recurrent neural network (RNN).

9. (canceled)

10. A system, comprising:
a view generator to create a plurality of volume rendered views of an organ of a patient;
a motion detector coupled to the view generator and including: a first network to extract spatial features from each input frame of the plurality of volume rendered views of the organ; a second network to extract temporal information from a sequence of volume rendered frames corresponding to the plurality of volume rendered views of the organ; and an algorithm to classify a severity of a motion abnormality of the organ; and
a display coupled to the motion detector to show the severity of the motion abnormality of the organ by assigning different colors to different levels of the severity of the motion abnormality of the organ.

11. The system of claim 10, wherein the plurality of volume rendered views of the organ includes a view showing a myocardial wall motion of the patient.

12. The system of claim 10, wherein the motion abnormality of the organ includes a regional ischemia or infarction.

13. The system of claim 10, wherein the motion abnormality of the organ includes a change in a cardiac (LV) function.

14. The system of claim 10, wherein the plurality of volume rendered views includes at least one of size, shape, or border zone of an infarct.

15. A method for detecting heart disease in a patient, comprising:
obtaining a plurality of volume rendering videos from cardiac imaging data of the patient;
classifying cardiac wall motion abnormalities present in the plurality of volume rendering videos; and
determining whether the cardiac wall motion abnormalities in the plurality of volume rendering videos are associated with the heart disease of the patient.

16. The method of claim 15, wherein the cardiac imaging data includes cardiac computed tomography (CT) data.

17. The method of claim 15, wherein the cardiac wall motion abnormalities include left ventricular (LV) wall motion abnormalities.

18. The method of claim 15, wherein determining whether the cardiac wall motion abnormalities in the volume rendering videos are associated with the heart disease of the patient includes:
extracting spatial features from each of input frames of the plurality of volume rendering videos;
synthesizing a temporal relationship between the input frames; and
generating a classification based on the extracted spatial features and the synthesized temporal relationship.

19. The method of claim 18, wherein the spatial features are extracted using a pre-trained convolutional neural network (CNN) configured to create N length feature vectors for each of the input frames, wherein N is a positive integer.

20. The method of claim 19, wherein the temporal relationship between the input frames is synthesized using a recurrent neural network (RNN) configured to include a long short-term memory architecture with N nodes and a sigmoidal activation function, wherein the RNN is configured to receive a feature sequence from the CNN and incorporate the temporal relationship.

21. (canceled)

22. The method of claim 18, wherein the classification is generated using a fully connected neural network, wherein the fully connected neural network is configured to estimate a severity of cardiac wall motion abnormalities in the plurality of volume rendering videos.

23. (canceled)

24. (canceled)
